(19) United States
(12) Patent Application Publication
(10) Pub. No.: US 2008/0306439 A1
(43) Pub. Date: Dec. 11, 2008

(54) Devices for mixing and applying a fluid composition

(76) Inventors: M. Bud Nelson, Raleigh, NC (US); Peyton Anderson, Raleigh, NC (US)

Correspondence Address: M. Bud Nelson;Affinergy, Inc., P.O. Box 14650, Research Triangle Park, NC, 27709-4650, US

(21) Appl. No.: 12/115,000

(22) Filed: May 5, 2008

Related U.S. Application Data
(60) Provisional application No. 60/942,755, filed on Jun. 8, 2007.
(60) Provisional application No. 60/991,400, filed on Nov. 30, 2007.

(51) Int. Cl. A61M 37/00

(52) U.S. Cl. 604/84

(57) ABSTRACT

An applicator device of the invention and a mixer applicator unit of the invention are each configured to enable mixing of two or more constituents to form a fluid composition. The applicator device comprises a syringe, a mixing chamber, and an applicator tip. The mixing chamber and applicator tip may be two separate components, or may be assembled into one integrated unit such as a mixer applicator unit of the invention. Provided in the applicator device or the mixer applicator are one or more fluid modifying elements for promoting mixing between two or more constituents. A reservoir is provided in the mixing chamber for storing a dry constituent in an embodiment wherein a constituent to be mixed comprises a dry constituent. The applicator device and mixer applicator unit each may further comprise a fluid control element for controlling the dispensing of a fluid composition from the applicator device or mixer applicator unit. Also provided with the present invention are kits comprising, as kit components, an applicator device (assembled or as separate component parts), and one or more accessories for use with the applicator device or mixer applicator unit, or combinations thereof.

### CROSS REFERENCE TO RELATED APPLICATIONS

This is a nonprovisional application which claims priority benefit of U.S. Provisional Application No. 60/942755, filed 8 Jun. 2007, and U.S. Provisional Application No. 60/99100, filed 30 Nov. 2007, which are hereby incorporated by reference herein.

### FIELD OF THE INVENTION

The present invention relates to an applicator device and a mixer applicator unit which promote mixing of two or more constituents, and more preferably a dry constituent with a fluid constituent, in forming a fluid composition. The fluid composition may then be controllably dispensed from the applicator device, such as to be applied to a substrate.

### BACKGROUND OF THE INVENTION

Often a composition that is comprised of two or more constituents is formed by mixing the two or more constituents immediately prior to use. For example, in the medical and dental fields, compositions such as cements, glues, and composite materials comprised of multiple constituents are prepared from their respective multiple constituents at the point-of-care (e.g., at the site of medical or dental application of the cement, glue, or composite material) just prior to use. Several systems have been developed for mixing and application of these compositions at the point-of-care; however, these systems have been developed primarily for mixing two or more fluid constituents in forming a fluid composition. For example, U.S. Patent Application Publication No. 20050209555A1 describes a syringe-to-syringe mixing system for mixing two fluids. U.S. Pat. No. 6,113,571 discloses an applicator device comprised of a double barrel syringe for mixing two fluid constituents to form a tissue glue, which may then be dispensed from the applicator device. U.S. Pat. No. 6,732,887 also discloses a syringe delivery system utilizing two barrels, wherein fluid constituents from each barrel are directed together through an applicator tip configured to mix the two fluid constituents, and dispensed is a resultant fluid. U.S. Pat. No. 5,489,266 discloses a syringe having a plunger assembly which enables lyophilization of an already formed liquid medication within the syringe barrel, and then the lyophilizate may be reconstituted only within the syringe by aspirating a diluent into the syringe barrel.

### SUMMARY OF THE INVENTION

It is an object of the invention to provide an applicator device for controllably applying a fluid composition, formed by mixing two or more constituents, onto a (at least one) surface of a substrate. The applicator device may comprise individual components that can be assembled to form the applicator device. The individual components can be detachably connected using fittings, connectors, or connecting configurations known in the art to include, but not limited to, Luer fittings (e.g., Luer-lock configuration, such as an external thread matingly cooperating with a an internal thread), snap-fit formations, friction fitting stem and slot formations, press-fit formations, tongue and groove formations, tab and slot formations, interlocking components, use of a non-permanent adhesive, or a combination thereof. Alternatively, the applicator device may be manufactured (e.g., molded, use of a permanent adhesive, or use of a lock fitting configuration) to include some or all of the functional components in a single, integral unit. According to one embodiment, the applicator device comprises a syringe device, a mixing chamber, a reservoir for the dry constituent, and an applicator tip. In a preferred embodiment, the applicator device allows use of a per se conventional syringe as a component of the applicator device, the component comprising the syringe device.

It is an object of the present invention to provide a device and system for: mixing two or more constituents, and preferably a dry constituent with a fluid constituent, in forming a fluid composition; and applying the fluid composition to a substrate.

Another object of the present invention is to provide an applicator device and delivery system, preferably for use at a point-of-care, which is specifically configured for mixing of a fluid constituent with a constituent in dry form to result in a fluid composition, and for dispensing the fluid composition from the applicator device. As will be described herein in more detail, the fluid composition may be dispensed through and from a dispensing tip of the applicator device.

In an embodiment where an applicator device or delivery system according to the present invention requires assembly of a plurality of components prior to use (e.g., at the point-of-care), also provided by the present invention is a kit comprising such components of the applicator device which are packaged together, such as in a single sterile container (e.g., box, tray, pouch, or other form of conventional packaging). The kit may also comprise a plurality of individually packaged components, and the individual packages can then be contained within a single larger container. The kit may additionally comprise instructions for use. For use in the medical field or dental field, preferably the components will be sterilized within the package or container so that they are immediately ready for use in a sterile environment.

Also provided according to the present invention is a mixer applicator unit comprising a mixing chamber and applicator tip, whether assembled or manufactured as a unit. The mixer applicator is configured to be securedly connected (e.g., either fixedly connected, detachably connected, or detachably connectable) to a syringe-like device (e.g., a syringe, a fluid transfer system, and the like) in a fluid tight engagement. In one embodiment, the syringe-like device may comprise a syringe prefilled with fluid constituent, and then the fluid constituent is dispensed from the syringe into a mixer applicator containing dry constituent, in promoting the mixing of fluid constituent with dry constituent in forming a fluid composition. In another embodiment, the syringe-like device may comprise a syringe or fluid transfer system which may contain dry constituent and fluid constituent which are separated (e.g., each in a separate chamber housed by the device, such a chamber may be present in the syringe body, plunger head, or a combination thereof), and are to be mixed immediately prior to use. In use, the syringe-like device is operated such that the fluid constituent and dry constituent are admixed in the syringe-like device, and then dispensed into the mixer applicator in promoting the mixing of fluid constituent with dry constituent in forming a fluid composition. In either embodiment, the fluid composition may then be dispensed from the mixer applicator tip in a desired flow and pattern of fluid which may include, but is not limited to, a stream, a spray, or other suitable fluid flow.

These and other benefits, advantages, and features of the present invention will become more fully apparent from the following description and appended claims.

### BRIEF DESCRIPTION OF THE DRAWINGS

While the present invention and specific embodiments described herein are illustrated in the following drawings, it is understood that these drawings are provided for a better understanding of the invention and use in practice, and are not therefore to be considered limiting of the scope of the present invention.

FIG. 1 illustrates a perspective view of one preferred embodiment of the applicator device of the invention that includes a syringe, and a unit comprising a mixing chamber and applicator tip (“mixer applicator”) attached to one end of the syringe.

FIG. 2 illustrates a perspective view of another preferred embodiment of the applicator device of the invention that includes a syringe, a unit comprising a mixing chamber and an applicator tip, and a vial adapter, wherein the components are shown as detachably connectable.

FIG. 3 illustrates a perspective view of an applicator device of the invention that includes functional and separate units: a syringe, a mixing chamber, an applicator tip, and an additional applicator tip; each unit shown as detached from the detachably connectable adjoining unit.

FIG. 4 illustrates a top perspective exploded view of one presently preferred embodiment of a mixer applicator unit for use with an applicator device of the invention; the mixer applicator unit comprising a 2-part outer housing, and an inner body member comprising a reservoir for a dry constituent.

FIG. 5 illustrates a bottom perspective view of the components illustrated in FIG. 4.

FIG. 6 illustrates a top plan view of one presently preferred embodiment of a mixer applicator unit for use with an applicator device of the invention, wherein the applicator device is in an “unactuated” position.

FIG. 7 illustrates a cross-sectional view taken along line A-A in FIG. 6.

FIG. 8 illustrates a perspective view of loading a fluid constituent into one preferred embodiment of the applicator device of the invention.

FIG. 9 illustrates a top plan view of one presently preferred embodiment of an applicator device of the invention being used to withdraw fluid constituent from a container.

FIG. 10 illustrates a cross-sectional view taken along line A-A in FIG. 9, and shows: the applicator device in an actuated position; the mixer applicator in an “open” position; and fluid flow fluid flow through the components of the applicator device.

FIG. 11 illustrates a top perspective exploded view of one presently preferred embodiment of a mixer applicator unit of the invention; the mixer applicator unit comprising a 2-part outer housing.

FIG. 12 illustrates a bottom perspective view of the components illustrated in FIG. 11.

FIG. 13 is a graph wherein columns labeled “A” and “B” depict the presence of a fluid composition formed in and applied by, either dropwise (“A”) or by spray mist (“B”), an embodiment of the applicator device depicted in FIG. 1; and an experimental control of fluid constituent alone (no dry constituent; “C”) applied by an applicator device of the invention.

### DETAILED DESCRIPTION OF THE PREFERRED EMBODIMENTS

The present invention provides for an applicator device and delivery system in which a non-liquid (“dry”) constituent can be contacted and mixed (e.g., such as in a process of reconstitution) with a fluid constituent to form a fluid composition at the site of the application of the fluid composition; e.g., mixing and application of such fluid composition at the point-of-care by a health professional. Mixing of a fluid constituent and dry constituent at the point-of-care or just prior to use may be important for those fluid compositions which lose their effectiveness and/or stability if stored for an extended period of time. A non-limiting example involves the use of a protein or peptide as a dry constituent which is reconstituted with a fluid constituent to form a fluid composition. In this example, the fluid composition is applied to a substrate such as a medical device, dental device, a tissue, or a combination thereof; e.g., for use in or on an individual in need of treatment with one or more of the fluid composition or a substrate to which with the fluid composition has been applied. For example, US Patent Application Publication No. 20060051395A1 (assigned to the present assignee) discloses a peptide-based biofunctional coating composition comprised of a surface-binding peptide (a peptide that binds to one or more of a metal, polymer, ceramic, or natural-based substrate (e.g., collagen-based) of which a device is comprised) coupled to a peptide having binding specificity for a growth factor. In continuing with this illustrative example, with respect to a surface of a substrate comprising one or more of a metal, polymer, or ceramic material, typically it is one or more surfaces of a medical device or dental device to which it is desired to apply the coating composition to form a coating thereon. In one embodiment, a coating composition is formed by mixing of the biofunctional coating composition with the growth factor for which it has binding specificity, and applying the coating composition to the intended surface.

By way of example, the fluid composition may comprise one or more of a biofunctional coating composition, medicament, or a composition desired to be applied to a surface of a substrate (biological or non-biological). Thus, the applicator device and delivery system are specifically configured to promote the mixing of a dry constituent with a fluid constituent in forming a fluid composition which then is dispensed from the applicator device. According to the invention, the dry constituent is preferably configured to be contained within, or placed in, the applicator device; and more particularly within a chamber which is not contained within a syringe-like device component (e.g., located external to the syringe component) of the applicator device.

A detailed description of the application device of the invention will now be provided with specific reference to figures illustrating preferred embodiments of the invention. It will be appreciated that like structures will be provided with like reference designations. To assist with understanding the scope of the invention, certain terms used throughout the application (including the specification and claims) are defined as follows.

The term “component”, as used herein, refers to a functional unit of the applicator device or delivery system or of the kit, regardless of whether the functional unit is a separate unit that is detachably connected or fixedly connected to another functional unit, or whether two or more functional units comprise a single integral unit. Each component of the applicator device or mixer applicator unit may be referred to by its general function (e.g., each as a “functional unit”). For example, a component may be selected from the group consisting of a syringe device, a mixing chamber, an applicator tip, and an adapter for securing a container and accessing a fluid contained therein.

The term “sufficient”, which is used in reference to mixing of two or more constituents, and more preferably a dry constituent with a fluid constituent, means adequate mixing to obtain a fluid composition with the desired characteristic. For example, a desired characteristic may include one or more of: reconstituting dry constituent with the fluid constituent; undergoing of a chemical reaction; curing in a desired manner; interacting with another composition, compound, or surface material of a substrate, in a covalent manner or non-covalent manner; and a combination thereof.

The term “fluid”, which is used in reference to a composition resulting from the mixing of two or more constituents in the applicator device or mixer applicator unit according to the invention, or in reference to a constituent which is used to form a fluid composition, means any flowable medium that may be suitably flowed through the applicator device or mixer applicator unit, and preferably refers to a liquid, a solution, or suspension.

The term “dry”, which is used in reference to a constituent which is used to form a fluid composition, means that the constituent comprises a form selected from the group consisting of a solid, semi-solid, powder, crystalline material, lyophilizate, particulate material, pellet, dry wafer, a material (e.g., microparticle, peptide, protein, nucleic acid, chemical compound, chemical composition, therapeutic agent, pharmaceutical agent, and the like) dried by any conventional means for drying as known in the art, a partially hydrated material that is not a flowing fluid (e.g., a gel), and a combination thereof.

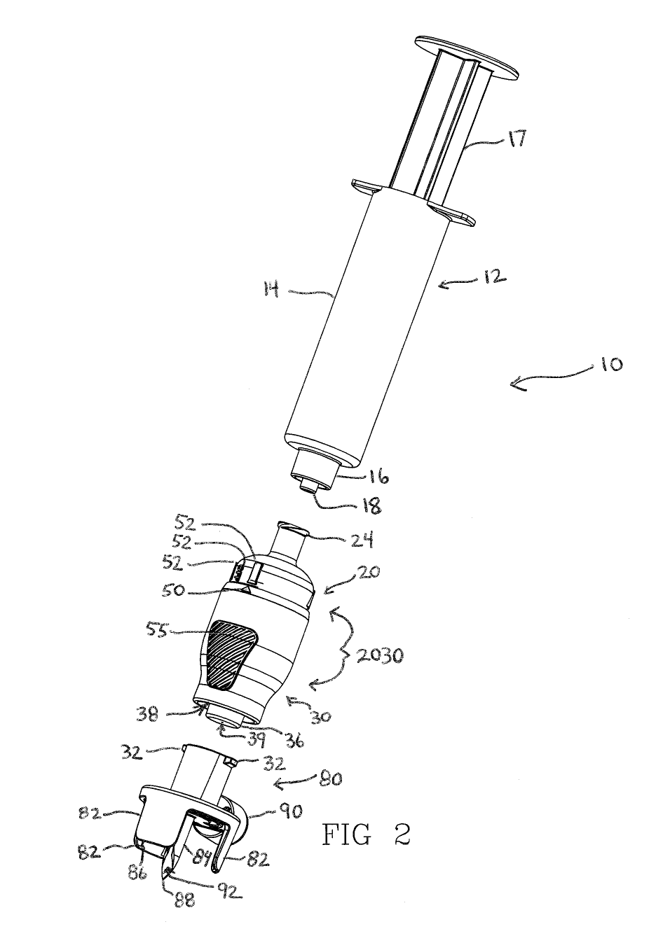

The term “connected”, which is used in reference to describing the relationship between two components of the applicator device or mixer applicator unit according to the present invention and unless otherwise specifically stated herein, means that the two components may be detachably connected or, alternatively, may be fixedly connected (e.g., the components are assembled or manufactured as an integral unit so that the two components cannot be detached). “Detachably connectable” means that the components are shown unconnected (see, e.g., FIGS. 2 & 3), but may be assembled (as described herein in more detail) to be detachably connected.

The terms “bottom”, “intermediate”, “top”, “distal”, “proximal”, “inner”, and “outer” are used herein for ease of description of component parts (“portions”) to be assembled, and are not to be construed to be otherwise limiting.

By way of introduction, the applicator device of the invention comprises a syringe-like device (“syringe”); a mixing chamber; a reservoir for holding the dry constituent (e.g., in which dry constituent may be placed or stored prior to contact with a fluid constituent); and an applicator tip. Optionally, the applicator device may further comprise an adapter for facilitating fluid access to a container from which is drawn the fluid constituent. A component of the applicator device is configured to be in a fluid tight engagement with any other component to which it is in fluid flow communication. As described in more detail herein and in a preferred embodiment, an applicator device may be comprised of two or more components, wherein adjoining components may be detachably connected, such as by detachable connectors. A non-limiting example of such detachable connectors capable of being mated to form a fluid tight engagement includes, but is not limited to, Luer fittings, a tongue and groove arrangement, snap-fit fittings, friction fitting, tab & slot fittings, or other detachable connection means known in the art. In another embodiment, wherein two or more components of the applicator device are fixedly connected, in the manufacture of the applicator device or components thereof, a molding process may be utilized to produce the components as one integral unit fixedly connected. Alternatively, the two or more components, manufactured as separate components, may then be glued, sealed, or mechanically locked together permanently, to be fixedly connected.

External to the syringe, and comprising components distinct from the syringe that may be assembled into or manufactured together as a unit, are the mixing chamber, a reservoir for holding the dry constituent, and an applicator tip. Thus, the reservoir does not comprise a portion of the barrel of the syringe, but rather is contained within a unit comprising one or more components external to the syringe. When the applicator device is in an open or actuated position, a passageway is provided from one end of the applicator device comprising the applicator tip (e.g., an aperture (one or more openings) in the applicator tip), through the mixing chamber, and into an opposite end of the applicator device comprising the barrel of the syringe. The passageway provides a fluid path that enables flow of fluid within and from the applicator device, such as between the syringe and the applicator tip. Also provided is another embodiment of the mixer applicator unit of the invention, wherein a mixer applicator comprises a mixing chamber and an applicator tip, wherein the mixer applicator contains one or more constituents to be mixed, and wherein the mixer applicator comprises a component distinct from, and located external to, the syringe-like device of the applicator device. Such mixer applicator may comprise components that are assembled into, or manufactured together as, a functional unit.

An applicator tip useful as a component of the applicator device of the invention or the mixer applicator unit of the invention may comprise any form or type, and be comprised of any suitable material for its intended purpose, through which a fluid can flow or be transmitted for application to a surface of a substrate. The applicator tip may include, but is not limited to, a spray applicator, a pad, a brush, sponge, rollerball, injection needle, cannula, and the like. With respect to a needle or needle-like cannula as an adapter that may be used with the applicator device, such adapter may also serve as an additional applicator tip. An applicator tip may be rigid, flexible, curved, straight, tapered, extended in length, or in some additional form, shape or geometry to accommodate application of the fluid composition to a surface to be treated. For example, an applicator tip may be flexed into or be a rigid, curved shape to accommodate application of the fluid composition to a surface of a substrate which is not easily accessible by a straight shape, the angle of such curve depending on the accessibility of the surface to be treated (e.g., such as surface of a tissue within a body cavity, or a surface of a medical device having inner recesses, as will be apparent to those skilled in the art).

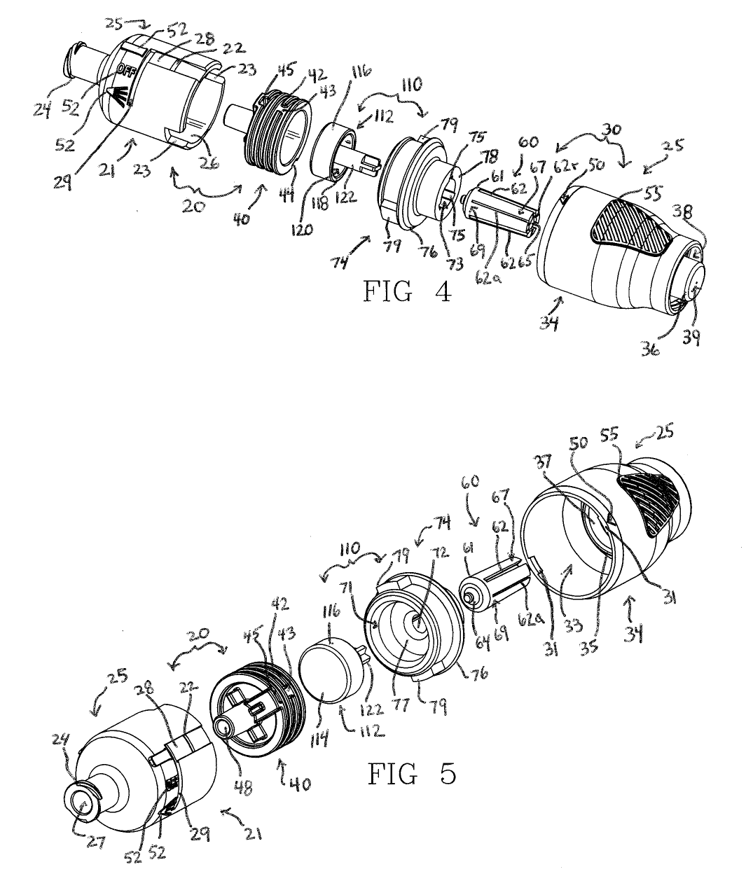

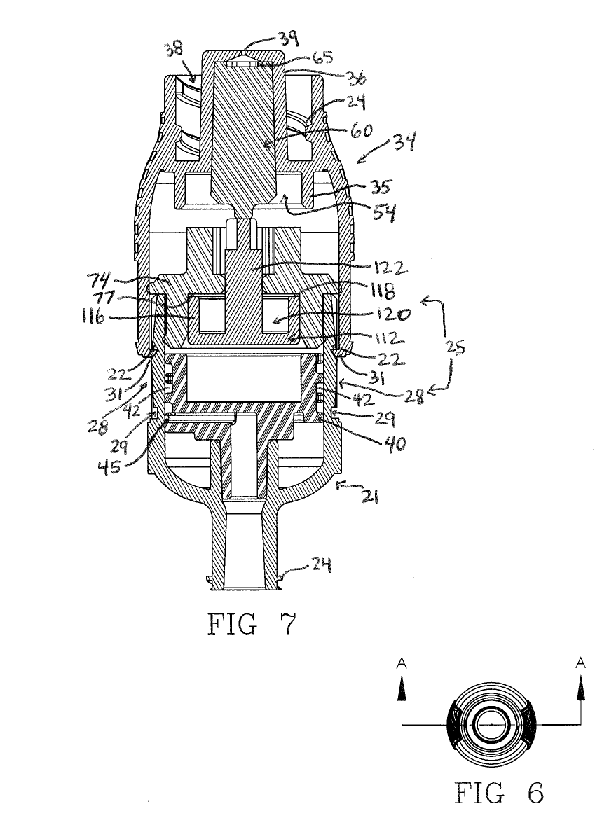

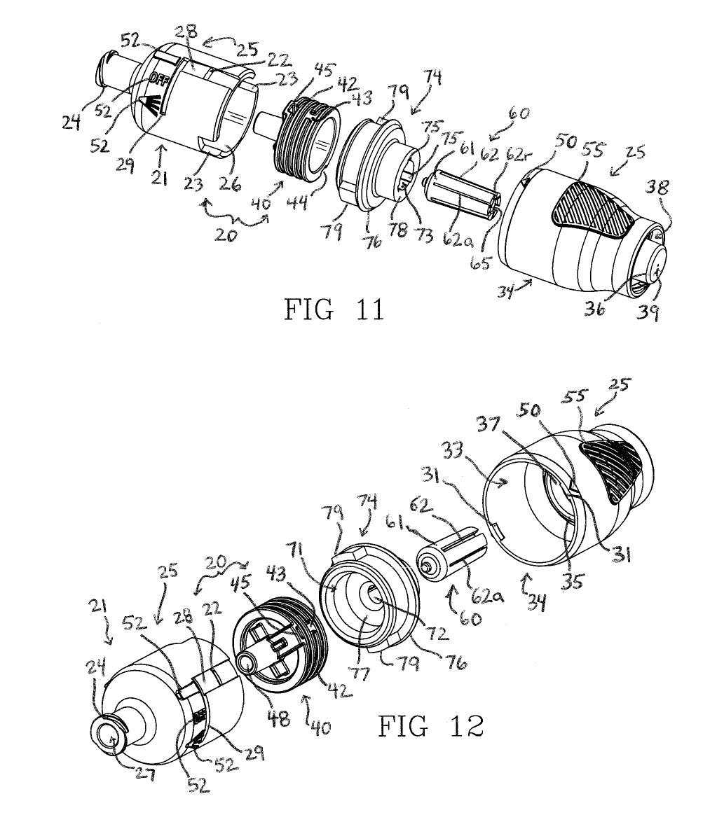

As will be described herein in more detail, as evident in FIGS. 4 & 5 showing an embodiment of applicator device of the invention and in FIGS. 11 & 12 showing an embodiment of the mixer applicator unit of the invention, preferably the applicator tip comprises a spray applicator. More preferably, provided is a unit comprising a combination of the mixing chamber component and applicator tip component in forming “mixer applicator” 2030, wherein the applicator tip comprises a spray applicator. Preferably, the spray applicator (by itself or as part of mixer applicator) has an adjustable spray pattern for adjusting one or more of the direction and level of atomization of the spray. Preferably, the spray applicator is configured to be capable of dispensing fluid composition in a form selected from the group consisting of a stream, a fine spray or mist, a spray pattern variable between a stream and a fine spray, and a combination thereof; and may further comprise an “off” position in which fluid composition is prohibited from being dispensed, so as to prevent dispensing accidentally. Except in special features specifically noted herein, it is further understood that the shape and design of a spray applicator to be used in an applicator device of the invention is generally not critical to the present invention, and thus could be substituted by other spray applicators as is conventionally known in the art. For example, the spray applicator may comprise a wand assembly comprised of a wand tube having at one end thereof a nozzle, and at the other end thereof a grip portion which may include a fluid control element (e.g., lever, or rotatable portion) for variably controlling the flow of fluid therethrough, and a hose connected at one end to the wand grip portion, and at the other end to the mixing chamber. A preferred applicator tip may be used in the invention to the exclusion of an applicator tip other than the preferred applicator tip.

In an embodiment wherein an applicator tip comprises a pad, a preferred suitable material may comprise a polymeric material through which may be transmitted a fluid, and more preferably, an open cell foam material. With a configuration of a brush or pad as a material and form of which applicator tip is comprised, fluid absorbed by or flowed through such material of the applicator tip could be applied to the surface of a substrate by contacting (e.g., pressing or brushing) this material with the surface of a substrate, and thereby transmitting the fluid onto the surface of the substrate to which is applied the fluid composition dispensed from the applicator device of the invention. Thus, for application of the fluid composition to a surface of a substrate, the applicator device is configured in an “open” position to enable fluid flow communication between the syringe and the applicator tip.

The applicator device further comprises a flow modifying element (one flow modifying element or, more preferably, a plurality of flow modifying elements) configured to affect the fluid flow in the fluid path in promoting the mixing of the at least two constituents (e.g., a dry constituent and a fluid constituent) to be mixed within the applicator device, wherein the mixing chamber or mixer applicator unit comprises at least one flow modifying element. A flow modifying element disrupts the fluid flow, such as by increasing the flow velocity, producing turbulence, eddies, a vortex, or other modification of fluid flow, that may improve fluid mixing of the at least two constituents. The flow modifying element may comprise one or more of a mechanical structure, or a configuration of a fluid path (e.g., configuration of the fluid flow channels or passageways which form the fluid path). For example, a mechanical structure known in the art to promote mixing of a fluid in a fluid path includes, but is not limited to, one or more of: a baffle, ridge, guide, vane, flute, or a combination thereof. When a flow modifying element comprises a mechanical structure, typically the flow modifying element is positioned (a) along a wall of, or surface internal to, or placed inside of, the applicator device which is in the fluid path, and (b) generally spatially orientated transverse to the direction of the fluid flow so as to disrupt the fluid flow in promoting mixing of the fluid flow. If desired, a flow modifying element can comprise circumferentially spaced, radially-orientated mechanical structures which are twisted along their lengths in a predetermined manner so as to impart a rotational force on the fluid flow in inducing a vortex mixing effect. In another example, an inner wall of the applicator device may be provided with inwardly projecting baffles or guides to direct fluid flow in a manner such as to promote mixing (e.g., by promoting vortex formation or turbulence).

Optionally, at any point within the applicator device, a configuration of the fluid path may be narrowed or constricted, or directionally altered in flow, so as to increase flow velocity and/or produce turbulence to promote mixing. By way of illustration, mixing may be promoted by narrowing or constricting the width of passageway as it extends away from mixing chamber and towards the syringe (e.g., wherein the narrowest portion is closest in proximity to the syringe component of the applicator device as compared to proximity to the mixing chamber). In another example, the fluid flow channels in the mixing chamber are formed in a helical configuration, thereby creating a vortex mixing of the fluid flow containing the at least two constituents to be mixed.

Because the fluid composition is formed within the applicator device or the mixer applicator unit, eliminated is the need for subsequent mixing upon dispensing the fluid from the applicator device or mixer applicator. Thus in accordance with the present invention, minimized are time, inconvenience, and mess as compared with current mixing processes which are external a device used for mixing or application. Mixing of two or more constituents, and preferably dry constituent with fluid constituent, preferably occurs in the mixing chamber of the applicator device or in the mixer applicator. More preferably, sufficient mixing of two or more constituents, such as but not limited to dry constituent with fluid constituent, in forming a fluid composition occurs in a component of the applicator device comprising the mixing chamber; e.g., by itself, or as part of a unit comprising the mixing chamber and applicator tip. For example, the fluid path through the mixing chamber is configured so that the reservoir, containing a dry constituent, is contacted by the fluid flow comprising a fluid constituent in initiating a mixing process that reconstitutes the dry constituent and promotes movement of the dry constituent out of the reservoir and into the fluid flow to be mixed with the fluid constituent by a flow modifying element comprising the mixing chamber. In an alternative embodiment, wherein the mixing chamber comprises a channel to direct fluid flow, such a channel may form a reservoir by depositing or otherwise storing a dry constituent in and along the channel or a portion thereof such that the dry constituent is in fluid communication with, and is contacted by, fluid flow comprising a fluid constituent aspirated into the applicator device. Mixing between dry constituent and fluid constituent occurs as the two or more constituents are contacted and moved in fluid flow in the mixing chamber. Thus, the mixing chamber, as a separate component or as part of a unit comprising mixer applicator, comprises a flow modifying element that promotes the mixing process. However, as apparent from the description herein, the applicator device may be configured so that one or more components, in addition to the mixing chamber or mixer applicator, may contribute to the mixing process as two or more constituents are mixed as they flow through that component. For example, as fluid comprising fluid constituent mixed with dry constituent is flowed from the mixing chamber and into the syringe device, further mixing than that occurring in the mixing chamber may be promoted as fluid is drawn into the barrel of the syringe with resultant turbulence, in a process of sufficient mixing in forming a fluid composition.

The applicator device of the invention may further comprise a fluid control element configured to regulate the fluid flow into the applicator device, or out of the applicator device, or a combination thereof. Also, the mixer applicator of the invention may further comprise a fluid control element configured to regulate the fluid flow into the mixer applicator, out of the mixer applicator, or a combination thereof. For example, a fluid control element may be in an “open” or actuated position wherein fluid flow is not restricted by means of the configuration of the fluid control element. Thus, in an “open” position, fluid may be introduced into or dispensed out of the applicator device of the invention or the mixer applicator of the invention. The fluid control element may be in the “closed” position, thereby completely restricting the fluid flow into or out of the applicator device or the mixer applicator. In such “closed” position, the fluid composition may be prevented from being accidentally dispensed from the applicator device, such as by pressure inadvertently applied to the plunger of the syringe device of the applicator device loaded with the fluid composition. Preferably, the fluid control element is configured such that the flow of the fluid into or out of the applicator device or mixer applicator is controllably variable to a selected rate. For example, the fluid control element may be configured to allow a degree of fluid flow variable between any position in a range of from a fully open position to a fully closed position. In another example, the fluid control element may be configured to provide a variable degree and variable function of fluid flow. As described in more detail herein, a preferred configuration of a fluid control element provides a fully open position or “stream” position, a fully closed position or “off” position, and an intermediate position or “spray” position. For purposes of clarity, a fluid control element is a component other than a conventional plunger of a syringe of the applicator device. However, a mechanism for locking or unlocking the movement of a syringe plunger or any other component of the applicator device, which mechanism functions to regulate fluid flow such as restricting or promoting fluid flow into or out of (depending on whether the mechanism is in the locked position or unlocked position, or in an open position or closed position) the applicator device, may also comprise a fluid control element.

In a preferred embodiment, and depending on the specific nature of the constituents being flowed or contained within applicator device, any one or more components of the applicator device, particularly those with one or more surfaces in contact with a fluid composition, should be comprised of a material that minimally reacts, or is substantially inert, with one or more of the fluid constituent, dry constituent, and fluid composition. For example, such a surface may be comprised of a material which does not react with a constituent or fluid composition in an undesirable way; e.g., such as an undesired chemical reaction between the surface and the fluid composition, or an undesired binding interaction between the surface and either the dry constituent or fluid composition in interfering with one or more of the formation of the fluid composition or the dispensing of the fluid composition from the applicator device. Such non-reactive materials are known in the art to typically include, but not limited to, polymers, or co-polymers. In a preferred example, such materials include, but are not limited to, polypropylene, and silicone. Additionally, in a preferred embodiment of the invention, the applicator device is disposable.

Optionally, one or more of the applicator device (or a component thereof) and the packaging for the applicator device may comprise an identifier. The identifier may take any form including a label (e.g., barcode, magnetic strip, laser-readable recognition pattern) applied on the applicator device or its packaging, a hardware or electronic identifier (e.g., microchip, radio frequency identification device) embedded in the applicator device or its packaging, or other suitable identifier readily readable or recognizable by a system usable at the point-of-care, such as a hand-held or stand-alone reader or recognition device, a computer terminal, a barcode scanner, or other suitable input device. Preferably, the identifier is in the form of a barcode label applied to the outer periphery of the applicator device or its packaging. The identifier can include information such as, but not limited to, any one or more of the composition, amount, lot number, and expiration date of the dry constituent loaded in the applicator device; instructions for mixing dry constituent with fluid constituent in forming a fluid composition; operating parameters of the applicator device; lot number of the applicator device; or a combination thereof. The system may upload the information communicated by the identifier so that an individual can track distribution of the applicator device, ensure that the applicator device selected for use contains the correct dry constituent for the intended use, track and record use of an applicator device to a specific individual (e.g., patient) receiving the fluid composition or medical device coated with the fluid composition, track and record removal of the applicator device from the vicinity of the patient (and may further include proper disposal of the applicator device), and a combination thereof.

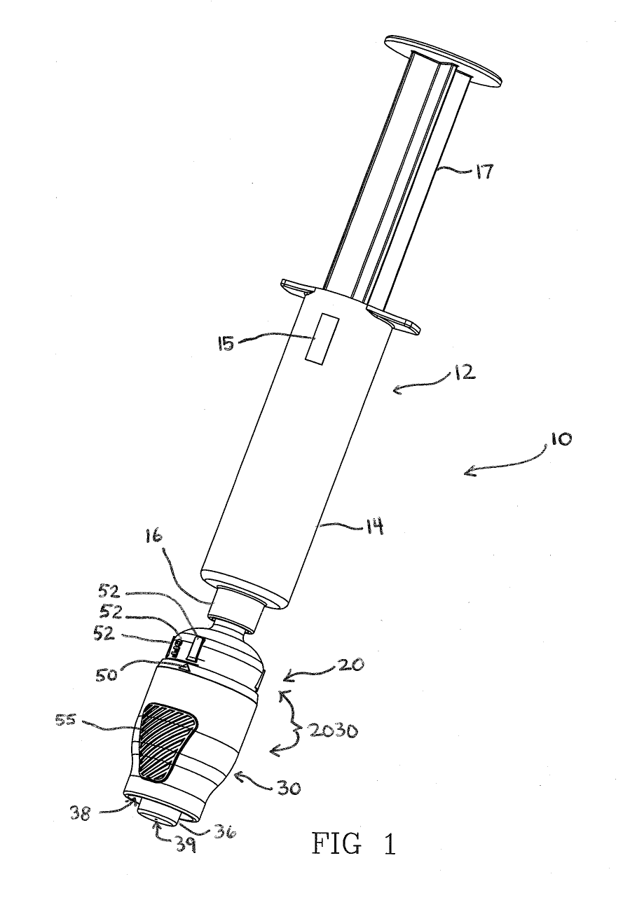

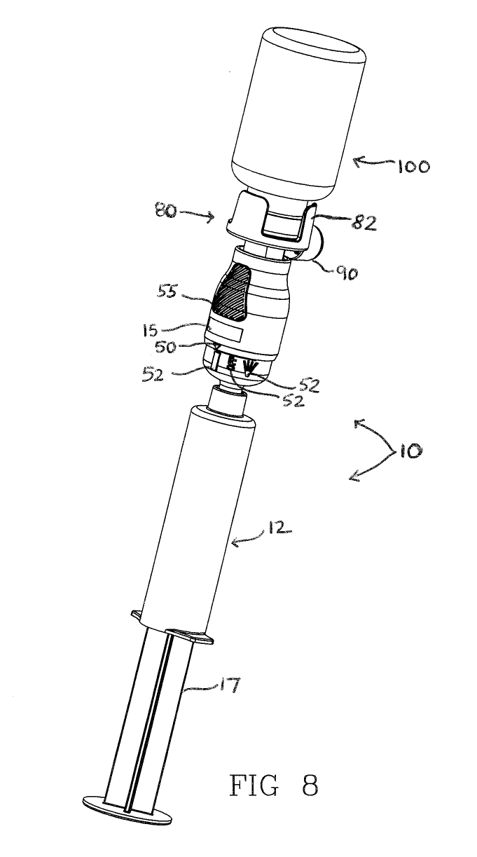

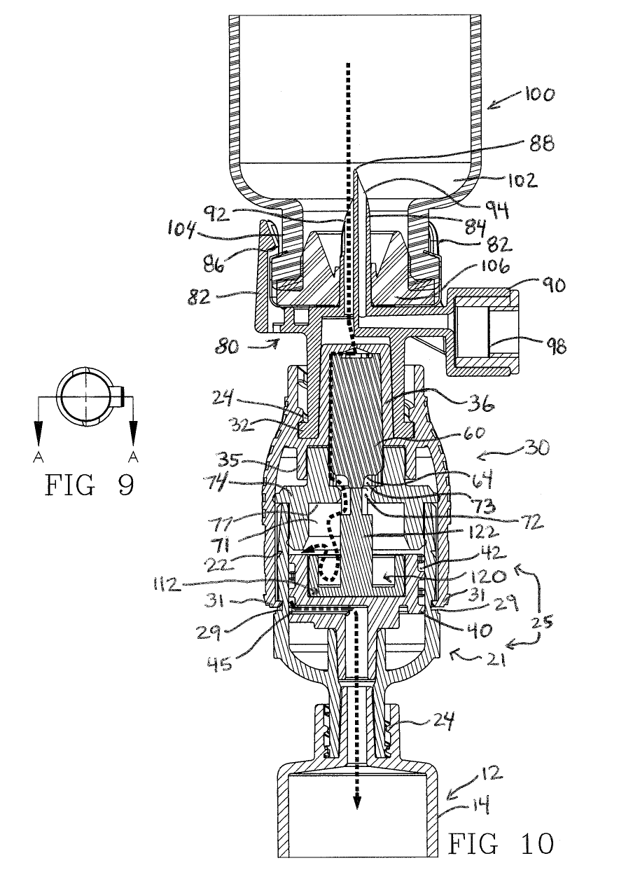

Referring now to the drawings, FIGS. 1-3 illustrate preferred embodiments of the applicator device 10 of the invention. As shown in FIGS. 1-3, and described in more detail herein, applicator device 10 comprises a syringe 12, a mixing chamber 20, and applicator tip 30. As shown in FIG. 2, applicator device 10 may further comprise adapter 80 which is used for transferring fluid constituent from a container and into applicator device 10. As shown in FIGS. 1 & 2, preferably, mixing chamber 20 and applicator tip 30 may be assembled together or manufactured to form a unit comprising both components as “mixer applicator 2030”. Mixer applicator 2030 may be fixedly connected to syringe 12 at one end thereof, or may be detachably connected to syringe 12 at one end thereof (see, e.g., FIG. 2) in a fluid tight engagement. In either case, enabled is fluid flow communication (or “fluid path”) between syringe 12 and mixer applicator 2030. Preferred embodiments of assembly of, or component parts comprising, mixer applicator 2030 are illustrated, for example, in FIGS. 4 & 5. In an embodiment wherein applicator device 10 comprises a component comprising an identifier 15, identifier 15 may be applied to or incorporated in applicator device 10 to be readily accessible to a system for reading or recognizing identifier 15. For purposes of illustration, and as shown in FIG. 1, identifier 15 is a label (such as a barcode) applied to the outer periphery of applicator device 10, and more specifically, to the outer surface of syringe 12. In another example, as shown in FIG. 8, identifier 15 is applied to the outer periphery of mixer applicator 2030, and more specifically, to an outer surface thereof.

Syringe 12, which may be provided as a commercially available syringe made of any conventional material, may comprise an elongated body typically referred to as a barrel 14; a piston device 17 which is typically referred to as a plunger; a syringe connector 16 typically comprising an annular recess which is threaded, such as a Luer fitting; and a fluid dispensing tip (see, e.g., FIGS. 2 & 3, fluid dispensing tip 18). Preferably, syringe 12 generally (a) is capable of drawing a fluid into applicator device 10, such as by drawing back on plunger 17 of syringe 12; (b) may further facilitate the mixing process, additional to the mixing of a dry constituent with a fluid constituent occurring in mixing chamber 20 or mixer applicator 2030, such as by turbulence of fluid flow as a fluid is aspirated into barrel 14 of syringe 12; and (c) facilitates dispensing of a fluid composition by pushing plunger 17 forward through barrel 14 of syringe 12 in expelling fluid out of syringe 12, through mixing chamber 20, through applicator tip 30, and out of applicator device 10. In one embodiment and as illustrated in FIG. 1, mixing chamber 20 by itself or as mixer applicator 2030, is fixedly connected to syringe 12. In another embodiment and as illustrated in FIGS. 2 & 3, mixing chamber 20 by itself or as mixer applicator 2030, is detachably connectable to syringe 12, such as by detachable connectors. A non-limiting example of such detachable connectors includes, but is not limited to, Luer fittings capable of being mated to form a fluid tight engagement, or other suitable fittings, connections, or connecting configurations.

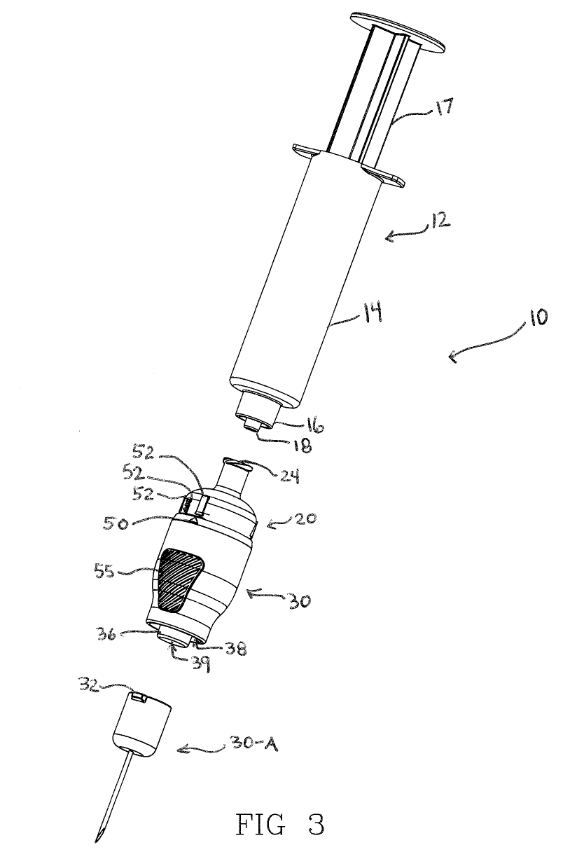

Optionally, applicator device 10 may further comprise an additional applicator tip 30-A which may be detachably connected to an end of either mixer applicator 2030 or applicator tip 30. In an embodiment wherein applicator tip 30-A is detachably connected to applicator device 10, mixer applicator 2030 or applicator tip 30 comprises a fitting that is configured to mate or connect with a fitting of applicator tip 30-A in a fluid tight engagement, and enabled is fluid flow communication between applicator tip 30-A and mixer applicator 2030. For example, with reference to one embodiment as illustrated in FIG. 3, applicator device 10 may further comprise an additional applicator tip 30-A which may be detachably connectable to an end of applicator tip 30 opposite to that by which applicator tip 30 is connected to mixing chamber 20. With reference to FIG. 3, one or more tabs 32 of applicator tip 30-A may be used to matingly cooperate with and securedly engage one or more fittings or slots (not shown) in annular recess 38 of applicator tip 30, in forming a fluid tight engagement between applicator tip 30 and applicator tip 30-A so that enabled is fluid flow communication between passageway 39 of dispensing tip 36 of applicator tip 30 and a passageway of applicator tip 30-A.

With reference to FIGS. 4, 5, 7, & 10, illustrated is a preferred embodiment of applicator device 10 in which mixing chamber 20 and applicator tip 30 are assembled or manufactured as a unit comprising mixer applicator 2030. In this illustration, mixer applicator 2030 comprises a 2-part outer housing 25 comprising a bottom portion 21 and a top portion 34. Bottom portion 21 may be configured to mate or connect with any syringe, in forming a fluid tight engagement between mixer applicator 2030 and syringe 12. In an embodiment wherein mixer applicator 2030 and syringe 12 are detachably connected, bottom portion 21 comprises fitting 24 that is configured to mate with syringe connector 16, and fitting 24 (and more preferably, passageway 27) is sized to receive fluid dispensing tip 18, in enabling fluid flow communication between syringe 12 and mixer applicator 2030. More specifically, as shown in FIG. 10, a fluid tight engagement is formed between passageway 27 of bottom portion 21 and passageway comprising fluid dispensing tip 18.

As shown in one embodiment of mixer applicator of the invention illustrated in FIGS. 4, 5, 7 & 10, and in a second embodiment of mixer applicator of the invention illustrated in FGs. 11 & 12, mixer applicator 2030 comprises a housing 25, preferably comprising bottom portion 21 configured to be connected with top portion 34. The assembly of bottom portion 21 with top portion 34 may be by any means known to those skilled in the art to include, but is not limited to, one or more of a friction-fit connection, snap-fit connection, threaded connection, and in any other suitable means to achieve the connection, and preferably in a fluid tight engagement. In a preferred embodiment, as shown in FIGS. 4, 5 & 7, and FIGS. 11 & 12, bottom portion 21 has one or more slots 28 for engaging one or more slot locking tabs 31 of top portion 34. As illustrated in FIGS. 4, 5, & 7, and FIGS. 11 & 12, optionally provided in slot 28 is an engaging lip 22 for engaging slot locking tab 31 so as to prevent slot locking tab 31 from reaching rotational guiding groove 29 and so as to secure top portion 34 comprising applicator tip 30 from functionally engaging bottom portion 21 comprising mixing chamber 20. As illustrated in FIG. 7, in this configuration utilizing engagement by engaging lip 22, the applicator device and mixer applicator are each in an unactuated position. By applying a sufficient amount of pressure on top portion 34 in a direction toward bottom portion 21 (such as, for example, from the downward force from inserting a container onto the adapter of the applicator device), overcome may be the engagement of slot locking tab 31 by engaging lip 22 so as to allow slot locking tab 31 to reach rotational guiding groove 29 in functionally engaging top portion 34 comprising applicator tip 30 with bottom portion 21 comprising mixing chamber 20. As illustrated in FIG. 10, in this configuration in which slot locking tab 31 reaches rotational guiding groove 29, the applicator device and mixer applicator are each in an actuated position.

Optionally, applicator device 10 or mixer applicator 2030 includes a component comprising one or more indicator markings associated with the housing comprising mixing chamber 20 and applicator tip 30 separately or as a unit comprising mixer applicator 2030. The indicator markings may be, but are not limited to, icons, logos, numbers, words, a notch, colors, shapes, or other visual cues that may be displayed to communicate information to the user of applicator device 10. For example, with reference to FIGS. 1, 2, 4 & 5, and FIGS. 11 & 12, the outer housing 25 of mixer applicator 2030 may be provided with a plurality of indicator markings. Indicator marking 50 disposed on top portion 34, illustrated in the form of a reference arrow or triangle, may be used to align with indicator markings 52 disposed on bottom portion 21. Indicator markings 52, disposed on bottom portion 21, may be positioned along and spaced apart relative to rotational guiding groove 29. Thus, when top portion 34 is rotated in relation to bottom portion 21 so that slot locking tab 31 moves along, and depending on its position in, rotational guiding groove 29, indictor marking 50 may be aligned with one of indicator markings 52 which provides information to the user as to a mode selected. For example, indicator markings 52 may provide visual cues as to the different modes permitted by a fluid control element, allowing a user to adjustably control and select a predetermined degree and/or pattern of fluid flow dispensed from the applicator device or from the mixer applicator. As illustrated in FIGS. 4 & 5, and FIGS. 11 & 12, indicator markings 52 preferably identify various positions selectable relative to fluid flow, such as a fully open position or “stream” position, a fully closed position or “off” position, and an intermediate position or “spray” position.

As illustrated in the embodiments depicted in FIGS. 4, 5, 7 & 10, and in FIGS. 11 & 12, provided in bottom portion 21 is mixing chamber 20 comprising a flow modifying element 40 which may be an integral part of bottom portion 21, a component connected to bottom portion 21, or a combination thereof. Preferably, the flow modifying element comprises static mixer 40. Static mixer 40 comprises an outer surface comprising a plurality of annular passages forming a channel for fluid flow which, with an outer wall for the annular passages, forms mixing channel 42. In a preferred embodiment, inner surface 26 of bottom portion 21 serves as the outer wall for the annular passages. As generally characteristic of static mixers, the mixing channel comprises a plurality of cross-channel openings distributed around the perimeter of the mixing channel, which openings function as feeder openings allowing the fluid to flow from one stage (e.g., annular passage) to another stage. The cross-channel openings promote mixing by allowing, at each stage, the stream of fluid flow to break into two streams of fluid flow traveling in opposite directions within a stage of the mixing channel until they join as one stream of fluid flow at the next cross-channel opening leading into the next stage of the mixing channel. For example, illustrated in FIGS. 4 & 5, and FIGS. 11 & 12, is cross-channel opening 43 in mixing channel 42.

As illustrated in FIG. 4, and in FIG. 10 by the segmented line with arrows showing a direction of fluid flow, fluid comprising fluid constituent and dry constituent is flowed from reservoir 110 into mixing channel 42 of static mixer 40 via feeder inlet 44 and into a first annular passage (“stage”) of mixing channel 42. The fluid flows around the first stage of mixing channel 42 until reaching first cross-channel opening 43, at which point the fluid splits into two fluid paths which travel in opposing directions in a second stage of mixing channel 42 until reaching a second cross-channel opening 43. At the juncture of the second cross-channel opening 43, the two streams of fluid mix first into one stream, and then separate again into two streams flowing in opposite directions when the fluid enters into the next stage of mixing channel 42. The static mixer 40 may comprise a mixing channel with a desired number of stages and cross-channel openings to provide sufficient mixing for the intended purpose. As shown in FIGS. 5 & 10, in the last stage of mixing channel 42, the fluid flow enters feeder outlet 45 and flows to discharge outlet 48. Fluid is flowed through discharge outlet 48 into passageway 27 of bottom portion 21 which is in fluid flow communication with passageway 18 of fluid dispensing tip 16 of syringe 12. Thus, features of static mixer 40 comprising the mixing of fluid streams in mixing channel 42, and the change in directional flow of fluid as it is flowed from the last stage of the mixing channel 42 and into the discharge outlet 48, promote mixing of fluid constituent with dry constituent in forming a fluid composition.

Provided in applicator device 10, and provided in an embodiment of mixer applicator 2030 as illustrated in FIGS. 4 & 5, is reservoir 110 for containing a dry constituent, the dry constituent intending to be mixed with fluid constituent in forming a fluid composition. Preferably, applicator device 10 is provided at the point-of-care with a predetermined amount and composition of dry constituent already loaded in reservoir 110. Alternatively, reservoir 110 may be provided in the form of a cartridge which is then loaded into applicator device 10 at the point-of-care and prior to use. Reservoir 110 may be provided in one or more components which may be assembled into or manufactured as reservoir 110. In reference to FIGS. 4 & 5 illustrating a preferred embodiment, reservoir 110 comprises reservoir tray 112 which may, when applicator device 10 or mixer applicator 2030 is in an unactuated position or mode, engage and cooperate with sealing element 74 to form a sealed chamber comprising reservoir 110. Reservoir tray 112 comprises a bottom 114 with an annular wall 116 extending therefrom and ending in face 118 in generally forming chamber 120, and may further comprise a rod member 122 that extends outwardly and centrally from the bottom of reservoir tray 112.

The sealing element 74 may be a gasket or comprise a form other than a gasket, such as an O-ring, mechanical seal, or other fluid sealing member, which is positioned between and in the engagement of bottom portion 21 comprising mixing chamber 20 with top portion 34, in providing a fluid tight engagement when assembled is outer housing 25. The sealing element or gasket 74 may be made of a resiliently yieldable material, such as a silicone rubber, synthetic elastomeric material, or other suitable material for forming a sealing arrangement that can also provide a fluid tight engagement between two or more components. Preferably, gasket 74 comprises a generally circular body, a flange portion 76, a lower chamber 71, an upper chamber 73, an opening 72 connecting lower chamber 71 with upper chamber 73, and one or more integrally molded tabs 79.

Referring to FIG. 7 showing the mixer applicator 2030 in an unactuated position or mode (or “closed” position), rod member 122 extends upwardly into and through opening 72 of gasket 74, with reservoir tray 112 snugly fitting into lower chamber 71, and with face 118 flush with a surface 77 of gasket 74 in forming a sealed chamber 120. Thus, a fluid tight seal is formed between reservoir 110 and gasket 74 which prevents fluid flow in a direction from applicator tip 30 to mixing chamber 20, and in a direction from mixing chamber 20 to applicator tip 30, in mixer applicator 2030 or in applicator device 10. Thus, in the unactuated position, reservoir 110 and gasket 74 cooperate to form a fluid control element configured to restrict the fluid flow into mixer applicator 2030 and into applicator device 10.

Referring to FIG. 10, mixer applicator 2030 is illustrated in an actuated position or mode (or “open” position). For example, as previously described herein, by applying a sufficient amount of pressure on top portion 34 in a direction toward bottom portion 21 (such as, for example, from the downward force from inserting a container 100 onto adapter 80 in operating applicator device 10), overcome is engagement of slot locking tab 31 by lip 22 so as to allow slot locking tab 31 and top portion 34 to slide toward rotational guiding groove 29 until slot locking tab 31 reaches rotational guiding groove 29. As top portion 34 moves toward rotational guiding groove 29, base portion 64 of spray nozzle 60 comes in contact with rod member 122 (of reservoir tray 112) which, prior to contact, is positioned in or extending slightly above upper chamber 73 of gasket 74. As top portion 34 of outer housing 25 is moved further toward rotational guiding groove 29, base portion 64 of spray nozzle 60 pushes rod member 122 in the same direction, in dislodging face 118 from engagement with a surface of gasket 74. As face 118 is no longer in engaging contact with gasket 74, absent is a fluid tight seal between face 118 and gasket 74; and thus, fluid flow is enabled through opening 72 of gasket 74 and in a direction from applicator tip 30 to mixing chamber 20 (e.g., see, FIG. 10, wherein the dotted line represents fluid flow), as well as in a direction from mixing chamber 20 to applicator tip 30, in mixer applicator 2030 or in applicator device 10. Thus, in the actuated position, reservoir 110 and spray nozzle 60 cooperate to form a fluid control element configured to promote the fluid flow into or out of mixer applicator 2030, and into or out of applicator device 10 (such direction depending on the selected operation of the applicator device by the user). In continuing with reference to FIG. 10, and as represented by the segmented line, as fluid is drawn into the mixer applicator 2030, the fluid will flow through applicator tip 30, through opening 72 into lower chamber 73 of gasket 74, and into chamber 120 of reservoir tray 112 to mix with and/or reconstitute dry constituent contained in reservoir tray 112.

In reference to FIGS. 4 & 5, and FIGS. 11 & 12, each tab 79 of gasket 74 is adapted to be received by and engage mounting slot 23 of bottom portion 21 of housing 25. The engagement of tab 79 with mounting slot 23 prevents unwanted rotational movement of gasket 74 when top portion 34 is rotated in relation to bottom portion 21, such as when selecting a mode or position relative to controlling or modifying fluid flow. Gasket 74, and particularly its flange portion 76, forms a fluid tight sealing arrangement between top portion 34 and bottom portion 21 of housing 25, wherein upper chamber 73 upwardly extends into top portion 34 and lower chamber 73 downwardly extends into bottom portion 21. More particularly, provided on an inner circumference of top portion 34 of housing 25 is annular channel 37 disposed on mounting member 35 within annular recess 33 of top portion 34. As shown in FIG. 7, mounting member 35 comprises an opening 54 configured to receive and engage spray nozzle 60. As shown in FIG. 5, surrounding opening 54 is annular channel 37 designed to receive and engage face 78 of upper chamber 73 of gasket 74 to provide a fluid tight seal when upper chamber 73 is inserted into and is receivingly engaged by annular channel 37, such as when mixer applicator 2030 is assembled or is moved into an actuated position. Thus, gasket 74 and annular channel 37 cooperate to provide a fluid tight seal between inner chamber of top portion 34 and gasket 74, as well as between top portion 34 and bottom portion 21 of housing 25.

In another embodiment of mixer applicator 2030, as illustrated in FIGS. 11 & 12, the mixer applicator comprises a mixing chamber 20, applicator tip 30, housing 25 for containing mixing chamber 20 and applicator tip 30, and sealing element 74; however, not included as a component of mixer applicator 2030 in this embodiment is reservoir 110. With reference to this embodiment of mixer applicator 2030, a reservoir for containing dry constituent is not needed when the dry constituent is contained within a reservoir or chamber which is located in a device other than in the mixer applicator; or two or more fluid constituents to be mixed an applied are in a device other than in the mixer applicator. For example, known in the art are devices comprising a prefilled syringe, or “multi-component syringe assembly” (having two or more chambers, each chamber containing a different constituent), or fluid transfer assembly, which devices contain at least two constituents each being physically separated from the other, and wherein at least one constituent is a fluid constituent (a constituent being either a dry constituent or fluid constituent). Non-limiting examples of such devices include a fluid transfer system as disclosed in U.S. Pat. No. 6,149,623 and published U.S. patent application US2007/0078428, in which a first constituent comprising a fluid constituent, and a second constituent comprising a fluid constituent or dry constituent, may be loaded into the syringe-like device; and a prefilled syringe, as disclosed in U.S. Pat. Nos. 4,599,082, 4,861,335, 5,899,981 and published U.S. patent application US 20060178638, which may comprise a syringe with two chambers which separate a fluid constituent and a dry constituent, or separate a first fluid constituent with a second fluid constituent, to be mixed immediately prior to use.

A mixer applicator unit according to the invention may be connected to such devices, in providing a means for sufficiently mixing a fluid constituent with a dry constituent, or sufficiently mixing two fluid constituents, to form a fluid composition immediately prior to use, as well as in providing a means for variably controlling one or more of the degree and function of fluid flow (e.g., such as by a fluid modifying element or applicator tip of choice). A preferred embodiment of a mixer applicator unit for this application is illustrated in FIGS. 11 & 12, wherein mixer applicator 2030 comprises a 2-part outer housing 25 comprising a bottom portion 21 and a top portion 34. Bottom portion 21 may be configured to mate or connect with a device such as a syringe or syringe-like device, in forming a fluid tight engagement between mixer applicator 2030 and the device. Having already described bottom portion 21 and mixing chamber 20 in reference to FIGS. 11 & 12, attention is now directed to the presence of a component comprising a sealing element, and to the absence of a reservoir tray disposed between the fluid modifying element such as static mixer 40 and sealing element 74. A sealing element may be in a form such as a gasket, O-ring, mechanical seal, or other fluid sealing member, which is positioned in the engagement of bottom portion 21 comprising mixing chamber 20 with top portion 34, in providing a fluid tight engagement when assembled is outer housing 25 of mixer applicator 2030. While the sealing element may be provided in various forms or configurations, preferably gasket 74 comprises form and function as previously described herein for gasket 74 while referring to FIGS. 11 & 12.

Provided is a method of using mixer applicator 2030 such as for mixing two or more constituents present in a syringe-like device selected from the group consisting of a pre-filled syringe, multi-component syringe assembly, and a fluid transfer assembly. Provided is a mixing and delivery system comprising the syringe-like device operatively connected to the mixer applicator unit. The syringe-like device may be packaged so that mixer applicator unit 2030 is pre-assembled with (already operatively connected to) the syringe-like device in forming the system. If not packaged in pre-assembled form, a first step is to assemble mixer applicator unit 2030 with syringe-like device by mating a connector of mixer applicator unit 2030 with a connector of the syringe-like device to form a fluid tight engagement in forming the system. Preferably, as part of the system, mixer applicator unit 2030 is initially in an “off” or closed position (as described herein in more detail) so as to prevent accidental dispensing from the system. Mixer applicator 2030 is then moved to an “on” or open position by the user so as to enable fluid flow through mixer application 2030, and to enable dispensing of fluid composition from mixer applicator unit 2030 in a predetermined degree and/or pattern of fluid flow. For example, top portion 34 is rotated in relation to bottom portion 21 so that slot locking tab may be aligned with indicator 52 identifying a position representing a particular fluid flow (e.g., a fully open position, or a variable degree thereof such as a “spray” position). Plunger of the syringe-like device of the system is then moved in the direction of the mixer-applicator unit to inject the constituents, contained within the syringe-like device, in a fluid flow through the syringe-like device and into mixer applicator unit 2030.

In reference to FIGS. 11 & 12, the fluid comprising more than one fluid constituent to be mixed, or comprising dry constituent and fluid constituent to be mixed, is dispensed from the syringe-like device through passageway 27 of bottom portion 21 which is in fluid flow communication with the syringe-like device. Fluid is flowed through discharge outlet 48 and enters feeder outlet 45 in entering mixing channel 42 of static mixer 40. The fluid flows around a first stage of mixing channel 42 until reaching a first cross-channel opening 43, at which point the fluid splits into two fluid paths which travel in opposing directions in a second stage of mixing channel 42 until reaching a second cross-channel opening 43. At the juncture of the second cross-channel opening 43, the two streams of fluid mix first into one stream, and then separate again into two streams flowing in opposite directions when the fluid enters into the next stage of mixing channel 42. The static mixer 40 may comprise a mixing channel with a desired number (preferably, a plurality) of stages and cross-channel openings to provide sufficient mixing for the intended purpose. The fluid is then flowed out from static mixer 40 via feeder inlet 44, through opening 72 of sealing element 74 and into applicator tip 30 to be dispensed out of dispensing tip 36.

In reference to the embodiments illustrated in FIGS. 4 & 5, and FIGS. 11 & 12 wherein applicator tip 30 comprises a spray applicator, in fluid communication with sealing element 74 is spray nozzle 60. Spray nozzle 60 generally comprises a spray nozzle body 61, and comprises a base portion 64, and a plurality of flow channels 62 or grooves extending longitudinally along the length or longitudinal axis of spray nozzle body 61 to spray nozzle port 65. Preferably, provided on spray nozzle 60 are two or more types of flow channels 62, each type generally differing from another type in width of the flow channel. For example, in a preferred arrangement or distribution on spray nozzle body 61 as illustrated in FIGS. 4 & 11, spray nozzle 60 comprises 6 flow channels 62, three of which are the same width and comprising a width narrower (62a) than the width shared by the other 3 (62); with the flow channels being distributed in an alternating arrangement relative to the radial axis of spray nozzle body 61 (e.g., 62, 62a, 62, 62a, etc.). Each generally longitudinal flow channel 62 preferably further includes a generally radial component (with respect to the longitudinal axis) 62r to the spray port 65.

In an embodiment illustrated in FIGS. 4 & 5, spray nozzle 60 is received by and securedly engaged in an opening in mounting member 35 configured to receive and engage spray nozzle 60, wherein a distal portion 69 (relative to dispensing tip 36) of spray nozzle body 61, including a portion of flow channels 62, extends into recess 33 to be in fluid communication with sealing element 74, and a proximal portion 67 (relative to dispensing tip 36) of spray nozzle 60, including a portion of flow channels 62, extends into the annular projection forming dispensing tip 36 to be in fluid communication with opening 39 of dispensing tip 36. In a preferred arrangement, spray nozzle 60 is fixedly secured in top portion 34 in forming applicator tip 30 or applicator tip portion of mixer applicator 2030. Thus, in this preferred arrangement and when mixer applicator 2030 is actuated, spray nozzle 60 rotates with top portion 34 as top portion 34 is axially rotated in relation to bottom portion 21 when slot locking tab 31 is axially moved along rotational guiding groove 29; whereas sealing element 74 remains in a fixed position due to engagement of tabs 79 with mounting slots 23. With reference to FIG. 4 and FIG. 11, upper chamber 73 of gasket 74 comprises an internal surface including a plurality of internal grooves or flow channels 75 which are spaced apart in relation to each other, and which extend longitudinally along the length or longitudinal axis of inner surface of upper chamber 73 including face 78. Thus, when mixer applicator 2030 is actuated and top portion 34 is axially rotated in relation to bottom portion 21, controllably varied is any alignment between flow channels 75 and flow channels 62. For example, if top portion 34 is rotated such that there is no alignment between flow channels 75 and flow channels 62, then distal portion 69 (a portion of spray nozzle body 61 other than comprising a flow channel) and annular channel 37 cooperate to provide a fluid tight seal, in preventing fluid flow from flow channels 75 of sealing element 74 into flow channels 62 of spray nozzle 60. This arrangement may constitute an “off” position or mode relative to the inability to dispense fluid flow in this arrangement; and an indicator marking 52 may be disposed on housing 25 to provide a visual cue to a user as to this mode in relation to fluid flow. In another example, if top portion 34 is rotated such that there is alignment between flow channels 75 and narrow flow channels 62a, then fluid flow is enabled from flow channels 75 of sealing element 74 into flow channels 62a of spray nozzle 60, and upon dispensing a fluid composition, a fine spray may be dispensed from opening 39 of dispensing tip 36. This arrangement may constitute a “spray” position or mode relative to fluid flow dispensed; and an indicator marking 52 may be disposed on housing 25 to provide a visual cue to a user as to this mode in relation to fluid flow. In yet another example, if top portion 34 is rotated such that there is alignment between flow channels 75 and wider flow channels 62 (as compared to the width of flow channels 62a), then fluid flow is enabled from flow channels 75 of sealing element 74 into flow channels 62 of spray nozzle 60, and upon dispensing a fluid composition, a streaming spray may be dispensed from opening 39 of dispensing tip 36. This arrangement may constitute a “stream” position or mode relative to fluid flow dispensed, and an indicator marking 52 may be disposed on housing 25 to provide a visual cue to a user as to this mode in relation to fluid flow. With each of applicator device or mixer applicator in an actuated position, rotation of top portion 34 with spray nozzle 60 alters which of its flow channels 62, if any, becomes aligned with, and in fluid communication with, flow channels of sealing element 74; hence, the control over, and enablement of, variability of the spray pattern and distribution.

Thus, a fluid control element of the applicator device of the invention, or of the mixer applicator of the invention, may comprise an ability to selectively enable continuity (be adjusted for alignment) or discontinuity (be adjusted for lack of alignment) between various flow channels disposed in mixer applicator 2030 (and hence, relative to enabling fluid flow or disabling fluid flow, respectively), and more particularly between flow channels provided in the sealing element in relation to flow channels of the spray nozzle. More preferably, spray applicator 30 (by itself or as part of mixer applicator 2030) comprises a fluid control element that is adjustable to be in an “off” or “closed” position (to prevent fluid composition from being accidentally dispensed from opening 39 of spray applicator 30, such as by pressure inadvertently applied to the plunger of a syringe), or in a variable “on” or “open” position to disperse a spray that can be varied from a fine mist to a stream spray (“adjustable spray pattern”) being dispensed from opening 39. The fluid control element may be provided (e.g., a portion of the spray applicator body such as top portion 34 may be rotated) to control the direction and level of atomization of the spray being dispensed from spray applicator 30, or to restrict dispensing of a fluid altogether, or a combination thereof. Optionally provided on top portion 34 is an external surface which is knurled to aid in gripping and controllably rotating that portion of the spray applicator. With reference to FIGS. 1-5, 8, and 11-12, illustrated is a preferred embodiment wherein on an external surface of applicator tip 30 is provided a gripping element 55 comprising a knurled (or pattern otherwise configured for ease of gripping) portion for ease of user to grip and hold applicator tip 30 in operating the applicator device or mixer applicator of the invention.

Optionally, as a component of the applicator device of the invention or as a component included in a kit comprising components comprising an applicator device of the invention and/or mixer applicator of the invention, provided is an adapter for facilitating the transfer of a liquid (e.g., fluid constituent) from a container and into the applicator device of the invention or mixer applicator of the invention. In a preferred embodiment, the spray applicator 30, by itself or as a portion of mixer applicator 2030, is configured and adapted to enable a fluid tight engagement with an adapter known in the art, or an adapter additionally provided (e.g., as a component of a kit according to the present invention); which adapter is suitable for connecting to a container (e.g., bottle, vial, ampoule, and the like) containing a fluid constituent. Connection between the adapter and container enables fluid flow communication between the container and the spray applicator (i.e., for aspirating the fluid constituent from the container and into one or more of the mixer applicator of the invention and the applicator device of the invention). In a preferred embodiment, adapter 80 is configured and adapted to be engaged with top portion 34 by detachable connectors in enabling fluid flow communication in a fluid tight engagement. A non-limiting example of such detachable connectors includes, but is not limited to, Luer fittings capable of being mated to form a fluid tight engagement, or other suitable fittings, connections, or connecting configurations. With reference to FIGS. 2 & 10, one or more tabs 32 of adapter 80 may be used to matingly cooperate with and securedly engage one or more fittings or grooves 24 in annular recess 38 of applicator tip 30 (by itself or as part of mixer applicator 2030), in forming a fluid tight engagement between applicator tip 30 and adapter 80 so that enabled is fluid flow communication between passageway 39 of dispensing tip 36 of applicator tip 30 and flow passage 92 of adapter 80. In addition to one or more tabs 32, adapter 80 comprises: one or more generally cylindrical side walls 82 extending from a base 83; a spike 84 having flow passages 92 and 94 extending parallel to each other, and terminating in a sharp point 88; and a venting element 90. Side walls 82 are configured to facilitate the positioning into adapter 80 of a container 100, such as a vial containing a liquid comprising a fluid constituent 102 to be aspirated into applicator device 10. Each side wall 82 may comprise a protuberance 86 projecting towards spike 84, wherein protuberance 86 engages neck portion 104 of container 100 to, coincident with side walls 82 and base 83, securedly hold container 100 in adapter 80. Spike 84 comprises flow passage 92 adapted to carry a fluid constituent from container 100, and comprises flow passage 94 adapted for the passage of air into container 100. In function, while fluid is being aspirated through flow passage 92, air is being passed through venting element 90 into flow passage 94 and into container 100. Thus, venting element 90 may provide a flow of air into container 100 without a user having to force air into container 100 prior to aspirating liquid comprising a fluid constituent 102 out of container 100. Venting element 90 is adapted to hold a filter 98. Filter 98 is designed to allow the passage of air. Preferably, filter 98 comprises a liquid non-permeable, gas permeable membrane; and more preferably comprises an anti-microbial filter which is standard in the art to be comprised of polymeric material with a pore size for excluding bacteria and airborne contaminants of similar size. Sharp point 88 is configured to puncture or lance penetrable closure 106 of container 100. Since fluid may be flowed in either direction through adapter 80, and adapter 80 comprises spike 84 having one or more flow passages through which fluid may be flowed, adapter 80 may also comprise an additional applicator tip 30-A, as previously described herein.

For use with the applicator device of the invention and the mixer applicator of the invention, the type and amount of the two or more constituents to be mixed in a method of mixing two or more constituents to form a fluid composition according to the invention are generally selected to address the desired result. Factors to consider for the desired result may include, but are not limited to, the nature of the fluid composition which is formed, the mode of application of the fluid composition to the surface, coverage area of the surface to which the fluid composition is to be applied, number of applications of fluid composition onto the surface, a concentration of the fluid composition effective for the function of the fluid composition per surface area of the surface to which it is applied, coating thickness formed from applying fluid composition to a surface, and the nature of the surface to which the fluid composition is to be applied.

Provided is a method of using the applicator device according to the invention. Applicator device 10 may be packaged so that two or more components comprising applicator device 10 are pre-assembled (already operatively connected in a fluid tight engagement). If not packaged in pre-assembled form, a first step is to assemble the components of the applicator device 10 by mating a connector of a component with a connector of an adjoining component to form a fluid tight engagement and until assembled is applicator device 10 (e.g., connecting syringe 12 with mixer applicator 2030, and then connecting mixer applicator 2030 to adapter 80; or connecting adapter 80 to mixer applicator 2030, and then connecting mixer applicator 2030 to syringe 12; see, e.g., FIG. 2). Optionally, upon unpackaging or any time thereafter, applicator device 10 or a component thereof having identifier 15 is presented so that identifier 15 is read by a system recognizing the information encoded by identifier 15. Preferably, as a component of applicator device 10, mixer applicator unit 2030 is initially provided in an unactuated position (as described herein in more detail, and illustrated in FIG. 7) so that fluid flow is not enabled throughout applicator device 10.

One step of the method according to the invention involves a “loading process” comprising aspirating fluid constituent from the container, which then flows through the adapter and components of the applicator device, and finally into the syringe of the applicator device where the liquid is contained within the syringe body. Typically, a fluid constituent 102 is stored in a container 100 such as a vial or ampoule. In referring to the embodiments of the invention illustrated in FIGS. 8 & 10, in use, container 100 is inverted and advanced toward adapter 80 to be engagingly received and held in place by adapter 80, such as in a snap-fit arrangement. In advancing container 100 in a downward direction toward adapter 80 to engage adapter 80, sharp point 88 and spike 84 pierces penetrable closure 106 of container 100. The downward force of securing container 100 into adapter 80 may be used to actuate mixer applicator 2030 and applicator device 10; e.g., the downward force being sufficient to (a) overcome engagement of slot locking tab 31 by lip 22 so that slot locking tab 31 slides to rotational guiding groove 29, and (b) base portion 64 of spray nozzle 60 pushes rod member 122 in dislodging face 118 from engagement with a surface of gasket 74 in enabling fluid flow through mixer applicator 2030; as previously described herein in more detail for deploying mixer applicator 2030 in an actuated position. With spike 84 in contact with the liquid contained in container 100 and with mixer applicator 2030 in an actuated position, enabled is fluid communication between container 100 and syringe 12 of applicator device 10. Plunger 17 is withdrawn out of syringe barrel 14 a distance sufficient to aspirate fluid constituent 102 out of container 100, through adapter 80, and into syringe barrel 14 of applicator device 10. In this process, fluid constituent 102 is mixed with dry constituent stored in reservoir 110 of applicator device 10, with mixing primarily in mixing chamber 20 or mixer applicator 2030 (see, e.g., FIG. 10) containing the dry constituent, whereby the dry constituent is reconstituted by the fluid constituent. The resultant liquid comprising fluid composition is contained in syringe barrel 14 of applicator device 10, and thus, the applicator device may then be considered in a “loaded” mode. It is noted that aspiration of the fluid constituent from container 100 may be aided by having a vented adapter 80 which allows air to be sucked into container 100 as fluid is aspirated out (see, e.g., FIG. 10). With applicator device 10 in loaded mode, adapter 80 and container 100 are detached from applicator tip 30 (itself or as part of mixer applicator 2030) of applicator device 10. This may be accomplished, for example, by detaching adapter 80 and container 100 as an assembly (while they are still connected together) by unmating detachable connector of adapter 80 from detachable connector of applicator tip 30. Alternatively, container 100 may be first removed from adapter 80 before unmating detachable connector of adapter 80 from detachable connector of applicator tip 30. In an option where adapter 80 is desired to be additional applicator tip 30-A, after disengaging container 100, connection between adapter 80 and applicator tip 30 may be maintained.

A next step of the method of using the applicator device according to the invention is referred to as a “dispensing” process. The dispensing process comprises discharging the liquid (contained in the syringe from the loading process) from the syringe, the liquid flowing through the applicator device until it is dispensed from the applicator tip of the applicator device. It is apparent that in the dispensing process, the liquid comprising fluid composition contained within the syringe will flow back through the mixing chamber or mixer applicator (depending on the embodiment of the invention) and may undergo mixing additional to the mixing that occurred during the loading process. Typically, mixing occurring in the loading process is sufficient to mix a dry constituent with a fluid constituent in forming a fluid composition. Some fluid compositions may require a short period of time to undergo a desired transformation (e.g., chemical or physical) prior to dispensing. Thus, in some circumstances and for certain constituents or fluid compositions, it may be beneficial to wait a predetermined amount of time (e.g., usually measured in minutes) before dispensing a fluid composition from a loaded applicator device. In the dispensing process, plunger 17 of syringe 12 is moved inwardly into syringe barrel 14 to discharge the liquid comprising fluid composition from syringe 12 and out of applicator tip 30 of applicator device 10. In a preferred embodiment wherein applicator tip 30 comprises a spray applicator, and more preferably mixer applicator 2030 comprises a spray applicator, subsequent to the loading process and prior to dispensing fluid composition out of applicator tip 30, the spray applicator may be controllably adjusted to provide the desired spray pattern and distribution of fluid composition to be dispensed from applicator tip 30, such as by a method previously described herein.

The method of the present invention may further comprise a step comprising an “application” process in which fluid composition dispensed from the applicator device is contacted with, and applied to, a surface which the user desires to have contacted by (e.g., applied or coated with) the fluid composition. Depending on the nature and type of applicator tip deployed on the applicator device according to the invention, contacting the fluid composition with the surface may comprise a process by which contact is mediated, the process selected from the group consisting of dipping, brushing, dabbing, rolling, spraying, streaming, injecting, or a combination thereof; or other suitable method for mediating contact between a liquid and a surface. For example, in a preferred embodiment wherein mixer applicator according to the invention comprises a spray applicator, the applicator device is held in proximity of the surface to be contacted, and fluid composition is dispensed through the spray applicator to the surface to be contacted. If desired, the spray applicator may be first adjusted for a predetermined spray pattern or distribution prior to the application process.

In another preferred embodiment of the present invention, provided is a kit comprising the applicator device of the invention or mixer applicator of the invention, and optionally may include kit components comprising accessories for use with the applicator device of the invention or mixer applicator of the invention. Such accessories may be selected from the group consisting of an adapter for a container containing a fluid constituent, one or more containers containing a constituent (e.g., a fluid constituent, or a constituent to be reconstituted to a fluid constituent), one or more additional applicator tips (which may vary in type; e.g., a brush or pad or needle or combination thereof may be included in a kit additionally to a spray applicator already provided with the applicator device, and which additional applicator tip may be detachably connected to one or more of mixer applicator 2030, mixing chamber 20, spray applicator 30, or syringe 12) a syringe-like device, instructions for use (of the applicator device or mixer applicator), a holder or stand for holding an applicator device, and a combination thereof. A kit comprises such components as an assembled unit, or as separated individual units or components thereof as described herein for assembly once unpackaged, or a combination thereof. The components in a kit may be packaged together, such as in a single sterile packaging container (e.g., box, tray, pouch, or other form of conventional packaging). The kit may also comprise a plurality of individually packaged components, and the individual packages can then be contained within a single larger packaging container. For use in the medical field or dental field, preferably the components will be sterilized within the package or packaging container so that they are immediately ready for use in a sterile environment upon unpacking.

### EXAMPLE 1

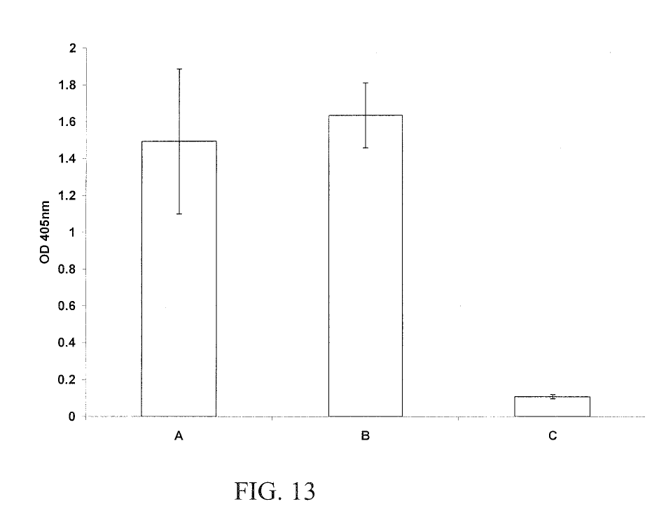

This example was designed to assess the mixing of a dry constituent with a fluid constituent to form a fluid composition using the methods and devices of the present invention; and to illustrate application of the resultant fluid composition to a surface using the applicator device. A dry constituent comprised a biotinylated peptide having binding affinity for a metal surface (e.g., stainless steel) was obtained in lyophilized (powder) form, and the dry constituent (approximately 1 mg) was loaded into and stored in the reservoir of the mixer applicator of the invention and the applicator device of the invention. The applicator device was then used to aspirate a fluid constituent comprising about 10 ml of a solution of phosphate buffered saline containing a detection conjugate (strepavidin-alkaline phosphatase). The fully loaded device was dispensed in one of two ways to apply fluid composition (a solution of metal-binding peptide labeled with alkaline phosphatase via the streptavidin-biotin interaction) to metal (stainless steel beads) contained in a 96 well microtiter plate (at a volume of about 200 μl per well). To illustrate use of the embodiment of the applicator device comprising an applicator tip comprising a spray applicator, the fluid composition was misted onto the metal beads via the spray applicator's spray mode. Also illustrated is the use of the applicator device, wherein a fluid composition is dispensed by being dripped from the orifice or dispensing tip of the applicator device onto the metal beads, such as from a stream mode. As an experimental control, the fluid constituent alone (i.e., absence of dry constituent) was added to the metal beads using a duplicate applicator device. All metal beads were then incubated for 30 minutes at room temperature, and then washed with buffer (Tris-buffered saline containing Tween™ 20). The metal beads were then transferred to individual wells of a new microtiter plate, and to each well was added 100 μl of color development reagent (“pNPP”; p-nitrophenyl phosphate), and subsequently the absorbance of solution from each well was recorded at 405 nm. FIG. 13 shows the absorbance measurements for a fluid composition formed in and dispensed from an applicator device of the invention, wherein the fluid composition is dispensed dropwise from the applicator device (FIG. 13, “A”) or via a spray applicator (FIG. 13, “B”), as compared to the experimental control (FIG. 13, “C”). As evident from FIG. 13, significant signal was detected for the presence of fluid composition (“A” and “B”), as compared to the experimental control (“C”). As shown in FIG. 13, illustrated is the use of an applicator device of the invention to provide sufficient mixing of a dry constituent (e.g., biotinylated metal-binding peptide) with a fluid constituent (e.g., a buffered solution of streptavidin-alkaline phosphatase) in forming a fluid composition (metal-binding peptide labeled with alkaline phosphatase). Further illustrated in FIG. 13, is the use of the applicator device of the invention to apply a fluid composition to a surface or substrate desired to be treated by the fluid composition.

The foregoing descriptions of the specific embodiments of the present invention have been described in detail for purposes of illustration. In view of the descriptions and illustrations, others skilled in the art can, by applying, current knowledge, readily modify and/or adapt the present invention for various applications without departing from the basic concept of the present invention; and thus, such modifications and/or adaptations are intended to be within the meaning and scope of the appended claims.

What is claimed is:

1. An applicator device comprising:
a syringe; and
a mixer applicator configured to be connectable or connected to an end of the syringe in a fluid tight engagement, wherein the mixer applicator comprises
(a) one or more fluid modifying elements, (b) a reservoir for storing a dry constituent, wherein in the reservoir is in fluid flow communication with a fluid path enabled in the mixer applicator, (c) a spray applicator, wherein the spray applicator is in fluid flow communication with a fluid path enabled in the mixer applicator, and (d) a fluid control element for enabling variable control over a fluid flow and spray pattern of fluid to be dispensed from the applicator device.

2. The applicator device of claim 1, wherein the spray applicator comprises one or more fluid control elements.

3. The applicator device of claim 1, wherein the syringe is detachably connected to the mixer applicator by use of detachable connectors.

4. The applicator device of claim 1, wherein the syringe is fixedly connected to the mixer applicator.

5. The applicator device of claim 1, further comprising one or more components selected from the group consisting of an adapter, an applicator tip, an indicator marking, an identifier, and a combination thereof.

6. The applicator device of claim 1, wherein the mixer applicator may be configured in a position selected from the group consisting of an unactuated position, and an actuated position; wherein in the unactuated position, fluid flow is prevented into or out of the mixer applicator; and wherein in the actuated position, fluid flow is enabled into or out of the mixer applicator.

7. The applicator device of claim 1, wherein the one or more fluid modifying elements comprises a static mixer.

8. A kit comprised of:
a syringe;
a mixer applicator configured to be connected or connectable to an end of the syringe in a fluid tight engagement and in fluid flow communication with the syringe in forming an applicator device, wherein the mixer applicator comprises
(a) one or more fluid modifying elements, (b) a reservoir for storing a dry constituent, wherein in the reservoir is in fluid flow communication with a fluid path enabled in the mixer applicator, (c) a spray applicator, wherein the spray applicator is in fluid flow communication with a fluid path enabled in the mixer applicator, and (d) a fluid control element for enabling variable control over a fluid flow and spray pattern of fluid to be dispensed from the applicator device; and
one or more accessories selected from the group consisting of an adapter for a container containing a constituent, a container containing a constituent, an applicator tip configured to be detachably connectable to the applicator device, instructions for use of the applicator device, a holder or stand for holding the applicator device, and a combination thereof.

9. The kit of claim 8, wherein the spray applicator comprises a fluid control element.

10. The kit of claim 8, wherein the syringe is detachably connected to the mixer applicator by use of detachable connectors.

11. The kit of claim 8, wherein the syringe is fixedly connected to the mixer applicator.

12. The kit of claim 8, wherein the applicator device further comprises one or more components selected from the group consisting of an indicator marking, an identifier, and a combination thereof.

13. The kit of claim 8, wherein the one or more fluid modifying elements comprises a static mixer.

14. A unit comprising a a mixer applicator, wherein the mixer applicator is configured to be connected or connectable to an end of a syringe-like device in a fluid tight engagement and in fluid flow communication with the syringe-like device in forming an applicator device, wherein the mixer applicator comprises one or more fluid modifying elements, and an applicator tip configured to be in fluid flow communication with a fluid path enabled in the mixer applicator.

15. The unit according to claim 14, further comprising a reservoir for storing a dry constituent, wherein the reservoir is in fluid flow communication with a fluid path enabled in the mixer applicator.

16. The unit according to claim 14, wherein the applicator tip comprises a spray applicator.

17. The unit according to claim 14, further comprising one or more components selected from the group consisting of an adapter, an applicator tip, an indicator marking, an identifier, and a combination thereof.

18. The unit according to claim 14, wherein the mixer applicator may be configured in a position selected from the group consisting of an unactuated position, and an actuated position; wherein in the unactuated position, fluid flow is prevented into or out of the mixer applicator; and wherein in the actuated position, fluid flow is enabled into or out of the mixer applicator.

19. The unit according to claim 14, wherein the one or more fluid modifying elements comprises a static mixer.

20. A kit comprised of:
a syringe-like device;
a unit comprising a mixer applicator configured to be connected or connectable to an end of the syringe-like device in a fluid tight engagement and in fluid flow communication with the syringe-like device in forming an applicator device, wherein the mixer applicator comprises (a) one or more fluid modifying elements, (b) a spray applicator, wherein the spray applicator is in fluid flow communication with a fluid path enabled in the mixer applicator, and (c) a fluid control element for enabling variable control over a fluid flow and spray pattern of fluid to be dispensed from the mixer applicator; and
one or more accessories selected from the group consisting of an adapter for a container containing a constituent, a container containing a constituent, an applicator tip configured to be detachably connectable to the mixer applicator, instructions for use of the mixer applicator, and a combination thereof.

21. The kit of claim 20, wherein the spray applicator comprises a fluid control element.

22. The kit of claim 20, wherein the syringe-like device is detachably connected to the mixer applicator by use of detachable connectors.

23. The kit of claim 20, wherein the syringe-like device is fixedly connected to the mixer applicator.

24. The kit of claim 20, wherein the applicator device further comprises one or more components selected from the group consisting of an indicator marking, an identifier, and a combination thereof.

25. The kit of claim 20, wherein the one or more fluid modifying elements comprises a static mixer.
